(19) United States
(12) Patent Application Publication
(10) Pub. No.: US 2003/0187458 A1
(43) Pub. Date: Oct. 2, 2003

(54) Correct surgical site marking system with draping key

(75) Inventor: Gerald I. Carlson II, Marietta, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/108,974

(22) Filed: Mar. 28, 2002

(51) Int. Cl. A61D 1/04; A61B 17/00

(52) U.S. Cl. 606/116

(57) ABSTRACT

A surgical site marking system for marking, correlating, and verifying that the surgical site which is to undergo the surgical procedure has been identified as the correct surgical site. The system utilizes labels and a series of checks to associate the patient with the correct surgery and appropriate surgical site.

### BACKGROUND

The present invention relates to a surgical site marking system and more particularly to a system for minimizing wrong-site surgical procedures.

Statistics show that medical errors are the eighth leading cause of death in the United States, accounting for 44,000 to 98,000 deaths each year. The measurable costs associated with medical errors are estimated to cost Americans nearly $37.6 billion per year. Non-measurable costs include loss of trust in the medical profession; diminished patient satisfaction; physical and psychological discomfort to the patient and the patient's family; and lower morale and increased frustration on the part of medical professionals themselves. Of these costs, nearly $17 billion per year are believed to be preventable. Prevention of these errors would naturally yield a commensurate positive impact on the non-measurable costs as well.

Some of these errors are attributable to communication breakdown; documentation errors; x-rays that are mislabeled, misread, and/or positioned incorrectly; chart errors; fatigue; impaired memory; pressure; and a lack of surgical site verification. The lack of surgical site verification often results in the occurrence of surgery being performed in incorrect locations. Moreover, it has been found that there is a statistically higher risk of incorrect or wrong-site surgery being performed in bilateral surgeries such as orthopedic surgery and the like.

To alleviate these problems it is desirable that a variety of approaches be considered. In addition, the goal is to combine these approaches with a coherent strategy and a devotion to error prevention on the part of the surgical team and hospital staff. Any system considered should be simple, easy to follow, capable of being standardized, and applicable to all surgical patient specialties.

Providing a simple system that helps minimize the occurrence of wrong-site surgical procedures is an approach that would be expected to go a long way toward meeting these goals.

### SUMMARY OF THE INVENTION

As such, one aspect of the present invention discloses a surgical site marking system including an incise material having an adhesive layer on one side for adhesion to a surgical site. The incise material is suitable for performing a surgical procedure therethrough by applying the incise material to the surgical site and performing the surgical procedure directly through the incise material itself. The incise material may be formed from a film, a mesh, or a combination of the two. The incise material also has an area adapted to receive data thereon. The data may be in the form of writing, an ink transfer, a decal, and/or a electronically scannable component, for instance, a bar code or computer chip.

Another aspect of the present invention is a surgical site marking system for use in a surgical procedure which contains an incise material which is adapted to be applied to a surgical site through which a surgical procedure is to be performed. The incise material may contain a film having an adhesive layer on one side and a removable material on the side opposite the adhesive layer. The adhesive layer may be provided with a releasable backing covering the adhesive layer until the incise material is readied for use. An area adapted for the recordal of data pertaining to the surgical procedure is also provided and may be placed on the removable material. The removable material enables the film portion, at the surgical teams' discretion, to remain sterile until the surgical procedure is performed.

In another aspect, the present invention is a surgical site marking system for use in a surgical procedure having a surgical drape with at least one fenestration and an adhesive backed layer of incise material keyed to the fenestration. The surgical procedure is to be performed through the incise material and fenestration.

Yet another aspect of the present invention provides for a method for identifying and verifying a surgical site on a patient prior to surgical incision. The method provides for labeling the surgical site with data pertaining to the surgical procedure to be performed and obtaining patient and surgical team verification that the correct surgical site through which the surgical procedure is to be performed has been labeled.

### BRIEF DESCRIPTION OF THE DRAWINGS

FIG. 1 illustrates the present invention in use on a patient's leg;

FIG. 2 illustrates an incise material in accordance with the present invention;

FIG. 3 illustrates another embodiment of an incise material in accordance with the present invention;

FIG. 4 illustrates still another embodiment of an incise material in accordance with the present invention; and

FIG. 5 illustrates use of an incise material in combination with a drape.

### DESCRIPTION OF THE INVENTION

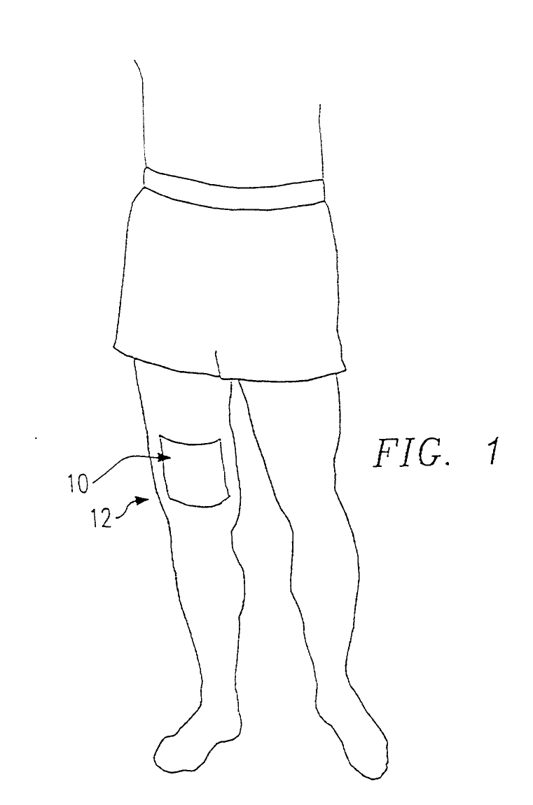

The present invention relates to a surgical site marking system for use in a surgical procedure. Proper use of the marking system enables one to accurately identify and verify the proper location of a surgical site prior to any incision. One embodiment of such a surgical site marking system according to the present invention is depicted in FIG. 1. FIG. 1 depicts an incise material 10 for placement on a patient surgical site 12. The incise material 10 may be formed of a sterilizable mesh, a membrane which may or may not be clear, an anti-microbial membrane, in addition to or in combination with other structures that allow a surgical procedure to be performed therethrough.

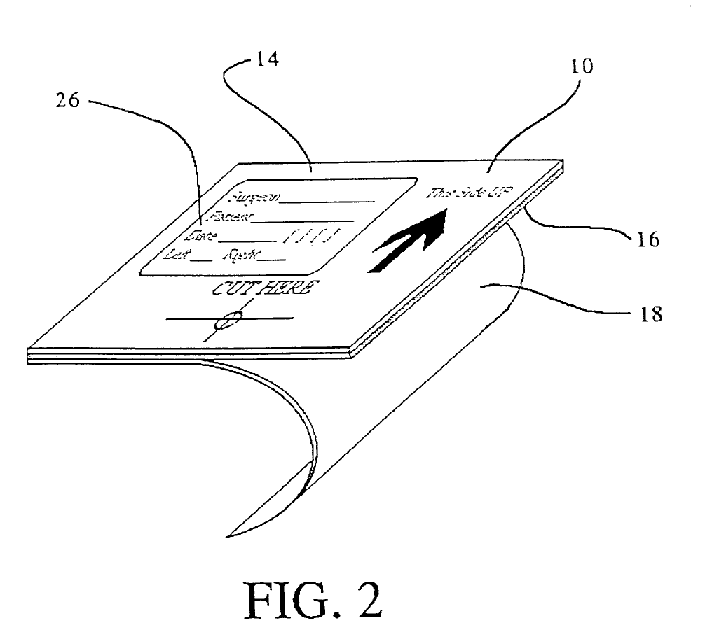

For example, as shown in the FIG. 2. embodiment, the incise material 10 may comprise a low-density polyethylene film 14 with an adhesive layer 16 on one side of the incise material 10 for adhesion to a surgical site. The film 14, may be clear, anti-microbial, and/or incorporate mesh. The adhesive layer 16 may be covered with a releasable backing 18. Such an incise material is available from Bertek Inc., St. Albans, Vt. 05478, or from Medical Concepts Development, Inc., St. Paul, N. Mex. 55125.

The releasable backing 18 may be formed of any of a wide variety of materials which are commonly available. For example, wax- or silicone-coated papers may be placed over the adhesive layer 16 of the incise material 10 which are removed when the incise material 10 is placed onto the patient surgical site 12.

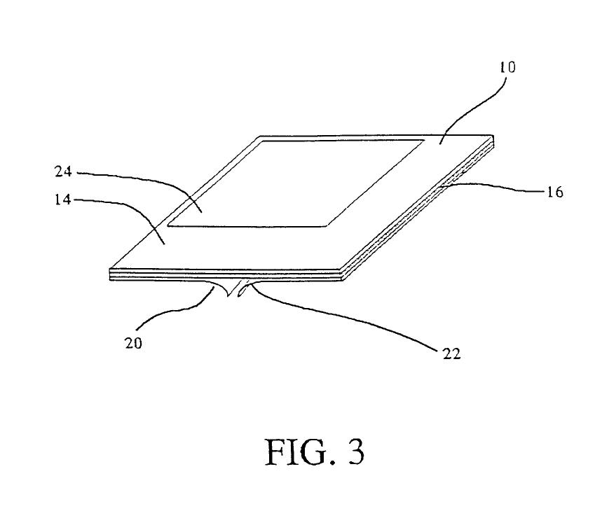

One possible alternative may be to provide the releasable backing 18 in the form of segmented and/or separate sections such as sections 20 and 22, as shown in FIG. 3. This embodiment may serve to facilitate application of the incise material 10 to the patient surgical site 12 or to make the releasable back sections 20, 22 easier to remove from the incise material 10.

Still looking to FIG. 3, it is shown that the film 14 side of the incise material 10 may contain a region or area 24 adapted to receive data thereon. The area 24 is adapted to receive data pertaining to the surgical procedure to be performed. Such data may include a surgeon's signature, the signature of the patient undergoing the procedure, and/or signatures of other members of the surgical team.

The area 24 may be adapted to receive the signatures and/or other data directly. Alternatively, still looking to FIG. 3 as shown, the area 24 may comprise a material or surface treatment disparate from the remainder of the film 14 so as to better adapt the area 24 to receive written data directly.

Now, looking back to FIG. 2, another possible embodiment envisions placing the signatures and/or other data on a label or decal 26 that in turn is affixed to the incise material 10, potentially in area 24. As such, the film layer 14 may comprise the area 24 (as seen on FIG. 3) which in turn may have a material or surface treatment disparate from the remainder of the film 14 so as to better adapt the area to receive the decal 26.

Additional data useful to identify the surgical procedure, the patient, and to verify the proper location of the surgery may also be provided on the incise material 10. This information could be located on the film 14, on area 24, on the decal 26, or on any combination of these. Possible useful data may include reference to the surgical procedure; may provide indicia for locating the incise material on the surgical site and/or provide indicia for locating surgical incisions; may refer to an appropriate surgical drape suitable for the procedure; corresponding custom procedure tray number; and/or drape pack number suitable for use with the procedure. Check boxes may be utilized as appropriate to allow designation of certain alternatives, such as left or right as shown on FIG. 2. In addition, or alternatively a series of check boxes may serve as a check list of tasks to be performed prior to performance of the surgical procedure.

Another possible embodiment (not shown) may provide for the application of an ink transfer pattern that may be transferred directly to the incise material 10, to the surgical site 12 itself, or to both. These ink transfer patterns may be utilized to contain some or all of the data discussed above.

Moreover, the marking system of the present invention may furnish the incise material 10 in sterile packaging. In some embodiments, the surgical site marking system may include a pen or other marking instrument (not shown). The pen may be sterile, but nevertheless would be suitable for marking on the incise material 10 or decal 26.

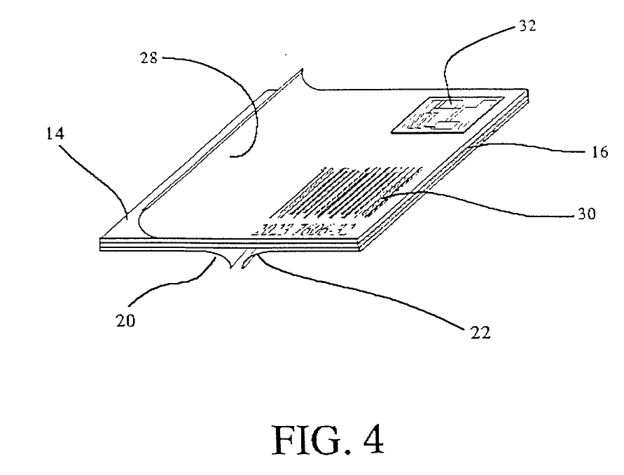

Another embodiment, shown in FIG. 4, depicts an additional layer or layers of material 28 which may be provided on the film 14 side of the incise material 10. This material 28 may cover all or a portion of the film 14. The material 28 may serve in its entirety or in part as the area where all data is entered. At some point prior to the actual surgical procedure, material 28 could be removed from the incise material 10, thereby exposing the film 14. This embodiment would preserve the sterility of the incise material 10, especially the surface of the film 14 until the surgical procedure.

In addition to, or in lieu of, area 24, decal 26, or material 28, other embodiments envision the use of electronically scannable components such as a computer readable code 30, a computer chip 32, and/or both as depicted on FIG. 4. In the case of computer readable or scannable components such as the UPC-, or bar-type computer readable code 30, and/or computer chip 32, scanning devices (not shown) are well-known that can read data from such components. Such a scanning device may be in the configuration of the pen already referred to above.

A number of possibilities exist for which this system may be appropriately utilized. In one manner, at least one member of the surgical team, for example the surgeon, consults with the patient being prepared for the surgery. The surgical team member confirms the location, for instance surgical site 12 in FIG. 1, of the surgery with the patient. At this point, the incise material 10 may be placed on the surgical site 12.

The patient, surgical team member, or both, would place their signatures or other confirmatory acknowledgement on the area 24 of the incise material 10 or the decal 26 which is subsequently adhered to the incise material 10 prior to or after placement of the incise material 10 on the surgical site 12. The surgery may take place under its normal course, providing the surgical team with a higher degree of assurance that the actual surgical site 12 has been verified.

In the event the incise material 10 includes material 28, similar acknowledgement and/or signature may be made in the appropriate locations. At some point just prior to the surgical procedure, the material 28 may be removed thus exposing the film 16 which would to that point remain in a sterile condition.

In the event that the patient is a minor or is otherwise incapacitated, verification and confirmation may be had with the patient's guardian or appointed representative. Alternatively, the system may be utilized unbeknownst to the patient. That is, verification and confirmation may be performed by and between the surgical team and hospital staff in the manner similar to that described above.

Of course, the system described is easily adaptable for use with electronic scanning technology. In addition to, or in lieu of handwritten confirmation, the use of bar code scanners and/or computer chip readers may serve to even more accurately correlate the patient's data to the surgical procedure to be performed.

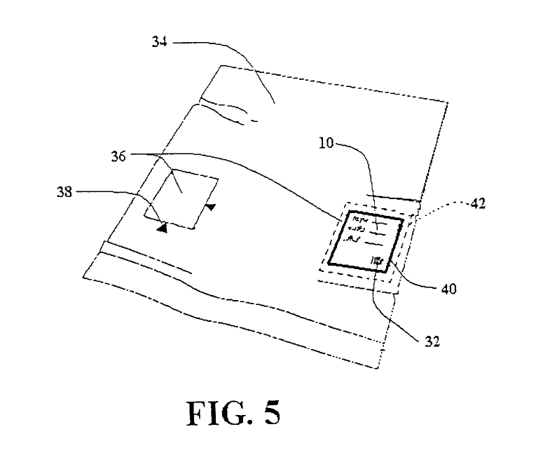

In another embodiment, as depicted in FIG. 5, the incise material may be packaged or otherwise designated to be used in conjunction with a specific surgical drape or drapes. In FIG. 5, the incise material 10 is shown in use with a surgical drape 34. The surgical drape 34 may contain at least one fenestration 36 associated with the incise material 10, however, additional fenestrations 36 may be provided in the drape 34 for possible use with additional incise materials 10. The incise material 10 may be manufactured and sold as a separate unit for use with a specific drape or drapes, or it may be sold as a part of a surgical pack including the drape 34.

By way of example, a surgical procedure may require more than one incision, such as in a heart bypass operation in which a patient's leg and chest are operated on. As stated above, specific incise materials 10 may be provided for use with each fenestration 36 in the drape 34. Alternatively, for bilateral-type surgeries such as arthroscopic knee surgery, a single drape 34 may be provided for simultaneously covering both knees. On these drapes, fenestrations 36 may be provided at each knee. Locating the incise material 10 at the correct surgical site 12 as described above will align the incise material 10 with the correct fenestration 36 through which the surgical procedure is to be performed.

In either case, as seen in FIG. 5, the incise material 10 may be keyed to the appropriate fenestration 36 in the drape 34. Keying the incise material 10 to the fenestration 36 may be accomplished in a variety of ways. Some examples include: the use of arrows or other registration indicia 38 to align the incise material 10 with the fenestration 36; matching the shape of the fenestration 36 with the shape of the incise material 10, aligning a border 40 on the incise material 10 with the fenestration 36; coding the incise material 10 to a particular fenestration 36 or drape 34; etc. Combining different aspects of these examples is also contemplated.

For example, FIG. 5 depicts both the use of registration indicia 38 markings placed upon the drape 34 at the fenestration 36 as well as the use of the border 40 on the incise material 10 aligning with the fenestration 36. It should be apparent that registration indicia 38 may also be placed on the incise material 10 to align with registration indicia 38 on the drape 34. In some embodiments, a perimeter 42, shown in phantom in FIG. 5, such as the outside perimeter of the incise material 10 may be matched to an appropriately sized fenestration 36.

Of course other means and manners of associating an incise material 10 with a fenestration 36 are envisioned and would be apparent to one skilled in the art. Coding of the incise material 10 to the fenestration 36 may also be readily adapted and accomplished through the use of data, either preprinted or electronically scannable such as through the use of the computer readable code 30 and/or computer chip 32. As such, the above enumerated list serves as an example of only a few such possibilities.

Use of the incise material 10 with a drape 34 initially is similar to use of the incise material 10 alone as described above. That is, data is associated between the patient, the surgical site and/or procedure, and surgical team and reflected via the data placed upon the incise material 10. The incise material 10 is applied to the surgical site 12 and verified by the patient, representative of the patient, and/or surgical team member per the above description.

In some embodiments, at this point, the drape 34 is placed in position over the patient. The fenestration 36 in the drape 34 is properly aligned with the incise material 10 as shown in FIG. 5. The result should be that the drape 34 is appropriately draped over the surgical site 12 with the incise material 10 located at the correct surgical site 12. In the event that the incise material 10 and the fenestration 36 do not align appropriately, this should signal to the surgical team prior to the procedure to once again verify that the correct surgical site 12 was labeled and that the correct drape 36 was being utilized for the procedure. Of course the patient may be draped with the drape 34 and the incise material 10 may subsequently be placed upon the patient.

In the event, that a bilateral surgery is to be performed, as depicted in FIG. 5, the drape 34 may be provided with a mirrored fenestration 36 at each surgical site, for instance, a matching fenestration 36 for each knee in a arthroscopic knee surgery drape. In this case, proper surgical site labeling and verification by use of the incise material 10 would result in one of the two fenestrations 36 aligning with the incise material 10 placed on the surgical site 12. The incise material 10 visibly present at the fenestration 36, and the existence of properly completed data on the incise material 10 provides validation to the surgical team that the surgical site 12, the incise material 10, and the drape 34 have been correlated.

In other embodiments, not depicted, the incise material 10 may be eliminated altogether and the surgical site 12 may be labeled by applying a visible pattern to the surgical site 12. The visible pattern may comprise an ink pattern transferred to the surgical site 12. Such an ink pattern may contain any portion or all of the data enumerated above and may also be endorsed as described.

Whether the invention includes a specific drape, a general drape, or no drape at all, means for labeling the surgical site 12 with data should help minimize the occurrence of wrong-site surgery. Such means for labeling may include: incise materials; labels, decals, mesh, membranes, films, patches, tattoos, inked patterns, ultraviolet patterns, projected images, electronically scannable components; and similar marking systems

The various embodiments described above are intended to describe possible aspects of the same invention. The elements described in each individual example are intended to be capable of substitution in whole or in part in any of the other examples. For example, the decal 26 may also be used with the material 28; the scannable code 30 and/or chip 32 could be in the form of the decal 26; the use of a drape 34 with an incise material 10 or the use of the incise material 10 alone may be utilized with any form of data, decal 26, and/or material 28; etc.

Furthermore, as used herein and in the claims, the term “comprising” is inclusive or open-ended and does not exclude additional unrecited elements, compositional components, or method steps. Additionally, as used herein and in the claims, the terms “a patient” or “the patient” refer to the particular patient undergoing the surgical procedure. Likewise the terms “a surgical team” or “the surgical team” refer to the specific surgical team performing the surgical procedure.

The invention may be embodied in other specific or equivalent forms without departing from the scope and spirit of the inventive characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A surgical site marking system for use in a surgical procedure comprising a surgical drape having at least one fenestration therethrough and an adhesive backed layer of incise material through which a surgical procedure is to be performed keyed to match the fenestration.

2. The system of claim 1 wherein the fenestration and incise material are shaped to match one another.

3. The system of claim 2 wherein the perimeter of the incise material matches the fenestration.

4. The system of claim 2 wherein a border located on the incise material matches the fenestration.

5. The system of claim 1 wherein the fenestration is shaped to register with registration indicia on the incise material.

6. The system of claim 1 wherein the fenestration is keyed with indicia which aligns with corresponding indicia on the incise material.

7. The system of claim 1 wherein the incise material is adapted to receive data recorded thereon.

8. The system of claim 1 wherein the incise material comprises a clear membrane.

9. The system of claim 7 comprising an electronically scannable component for receipt and review of data.

10. The system of claim 1 wherein the incise material comprises an area adapted to receive signatures.

11. The system of claim 1 comprising a label for application onto the incise material.

12. The system of claim 11 wherein the label comprises an ink transfer adapted to be applied to the incise material.

13. The system of claim 1 wherein the incise material comprises data pertaining to any of surgical procedure type, incision locator, corresponding drape product code, corresponding custom procedure tray number, and drape pack number.

14. A draping system for a surgical procedure comprising a fenestrated surgical drape suitable for performing an intended surgical procedure and means for labeling a surgical site, the means keyed to an appropriate fenestration in the drape through which the surgical procedure is to be performed.

15. The system of claim 14 wherein the means for labeling the surgical site comprises an incise material.

16. The system of claim 14 wherein the means for labeling the surgical site comprises a pattern transfer.

17. The system of claim 16 wherein the pattern transfer is an ink transfer.

18. The system of claim 14 wherein the means for labeling the surgical site comprises a decal.

19. The system of claim 14 wherein the means for labeling the surgical site is adapted to be signed by at least one party involved in the surgical procedure.

20. The system of claim 14 wherein the means for labeling the surgical site comprises indicia directing proper registration of the fenestration in the drape with the means for labeling.

21. The system of claim 14 wherein the means for labeling the surgical site comprises data pertaining to any of surgical procedure type, incision locator, corresponding drape product code, corresponding custom procedure tray number, and drape pack number.

22. A surgical draping system for use in a surgical procedure comprising a surgical site label for application on a surgical site through which an intended surgery is to be performed; and a surgical drape having at least one fenestration therethrough, the fenestration keyed to match with the surgical site label.

23. The system of claim 22 wherein the surgical site label is inked onto the surgical site.

24. The system of claim 22 wherein the surgical site label comprises any combination of incise material, a decal, a film, a mesh, a patch, and a tattoo.

25. The system of claim 22 wherein the surgical site label is adapted to be signed by at least one party involved in the surgical procedure.

26. The system of claim 22 wherein the surgical site label comprises indicia directing proper registration of the fenestration in the drape with the label.

27. The system of claim 22 wherein the surgical site label comprises data pertaining to any of surgical procedure type, incision locator, corresponding drape product code, corresponding custom procedure tray number, and drape pack number.
